(19) United States
(12) Patent Application Publication
(10) Pub. No.: US 2008/0242636 A1
(43) Pub. Date: Oct. 2, 2008

(54) MODIFIED CYCLODEXTRIN SULFATE-71 (TERIGYN) AS A MICROBICIDE AGENT

(76) Inventor: Elijah Martim Songok, Winnipeg (CA)

Correspondence Address: ADE & COMPANY INC., 2157 Henderson Highway, WINNIPEG, MB, R2G1P9, CA

(21) Appl. No.: 12/054,749

(22) Filed: Mar. 25, 2008

Related U.S. Application Data
(60) Provisional application No. 60/908,226, filed on Mar. 27, 2007.

(51) Int. Cl. A61K 31/724; A61P 31/18

(52) U.S. Cl. 514/58

(57) ABSTRACT

A method of reducing human immunodeficiency virus infective events by applying terygyn and a suitable excipient formulated for topical administration to the vaginal, anal, urethral or penile glans mucosa of an individual prior to or subsequent to infection by human immunodeficiency virus is herein described.

### PRIOR APPLICATION INFORMATION

The instant application claims the benefit of U.S. Provisional Patent Application 60/908,226, filed Mar. 27, 2007.

### BACKGROUND OF THE INVENTION

Despite major worldwide efforts to develop a microbicide, their number in clinical trials is very limited and none, so far has been approved for marketing. Large scale efficacy trials for five products, involving tens of thousands of women and tens of millions of dollars are either planned or underway. Three of these products PRO2000, Carraguard and cellulose sulfate are anionic polymers and inhibit HIV-1 infection by preventing virus-cell fusion predominantly through charged based interactions. In addition, Viva Gel (SPL7013, a sulphated dendrimer) thought to work through similar mechanisms has been entered in early phase I safety trials. The fourth product in phase III trials is a buffer gel (Buffergel) containing polyanionic caborpol while the fifth is based on the novel surfactant C31G (termed SAVVY). These agents inhibit both HIV and HSV in cell culture and animal models.

From the above, it is evident that anionic or polyanionic compounds are the most likely candidates to be approved as microbicides. However studies have shown that all the above require more than 10-fold more compound to block infection with HIV CCR5 tropic viruses (R5) than with CXCR4 (X4). As R5 viruses are the first to establish infection in a host, there is a need for more potent versions of polyanionic compounds to be considered alongside the first generation microbicide candidates.

### SUMMARY OF THE INVENTION

According to a first aspect of the invention, there is provided a method of reducing human immunodeficiency virus infective events comprising: providing a pharmaceutical composition comprising an effective amount of terygyn and a suitable excipient formulated for topical administration; and applying the pharmaceutical composition to the vaginal, anal, urethral or penile glans mucosa of an individual prior to or subsequent to infection by human immunodeficiency virus.

According to a second aspect of the invention, there is provided a pharmaceutical composition comprising an effective amount of terygyn and a suitable excipient for topical administration.

### BRIEF DESCRIPTION OF THE DRAWINGS

FIG. 1. Steps in the synthesis of mCDS-71 (Terygyn).

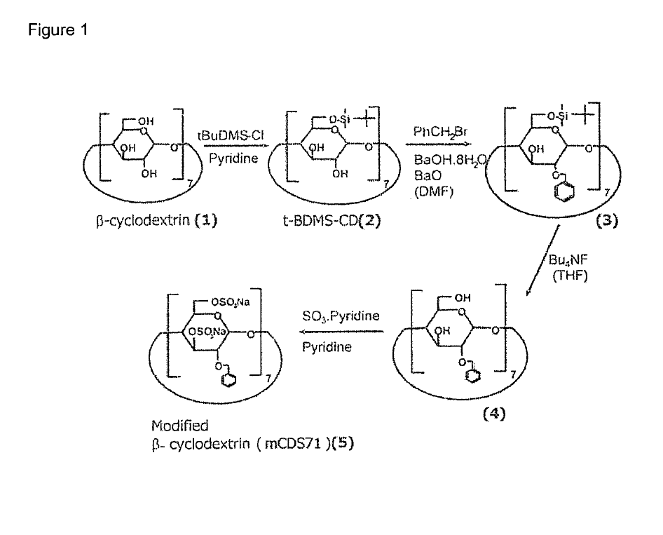

FIG. 2. The general framework of mCDS compounds as depicted by mCDS-11.

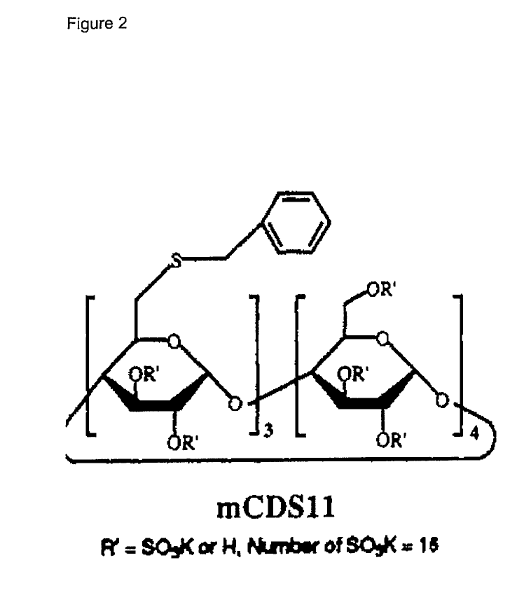

FIG. 3. Effect of terygyn on apoptosis on CD4+ cell subsets.

FIG. 4. Effect of terygyn on CD8+ cell subsets.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Terigyn (m CSD71, Tetradecasodium heptakis (2-0 benzyl)β-cyclodextrin tetradecasulfate,) a modified polyanionic compound, was developed and initially described as m CSD71 by Tanabe Seiyaku company limited of Osaka Japan in 1991 as an oral anti-HIV agent. It showed anti-HIV activity in vitro of 0.98 ug/ml and safety trials on rats showed a tolerability of 125 mg/kg/day.

For oral administration, the drug or agent is placed in the mouth and swallowed for it to have the desired anti-HIV activity. Furthermore, the oral administration was prescribed for treatment of HIV infection after the virus has already entered the cells of the body and had started to cause or were about to cause the disease AIDS. The inventor believes that when terygyn is taken orally, it is poorly absorbed in the intestinal and stomach mucosa, thereby reducing its potency. The inventor proposes that to increase the potency of terygyn, it will need to be applied both orally and topically to reduce the number of infective HIV agents.

Described herein is the topical administration of terygyn for reducing the rate of HIV infection or the absolute number of infective events compared to an untreated control, that is, an HIV infective event wherein terygyn has not been applied topically. It is of note that topical application in this case refers placing an effective amount of terygyn in a pharmaceutically acceptable carrier for topical administration in direct contact with or onto a spot, which can be in or on any part of the body, including but by no means limited to the epidermis, any other dermis, or any other body tissue. Preferred areas for topical administration of terygyn include vaginal, anal, urethral or penile glans mucosa and accordingly in a preferred embodiment there is provided a pharmaceutical composition for use in reducing HIV infective events in vaginal, anal, urethral or penile glans mucosa compared to infection with a similar number of HIV particles on untreated vaginal, anal, urethral or penile glans mucosa. As discussed herein, topical administration blocks viruses from entering the cells and causing infection. It may be applied both prior to infection to reduce the risk of infection and/or after infection to reduce the spread of infection to susceptible cells. Treatment times may be before or immediately after suspected infection.

In another embodiment of the invention, there is provided a method of reducing human immunodeficiency virus infective events comprising: providing a pharmaceutical composition comprising an effective amount terygyn and a suitable excipient formulated for topical administration; and applying the pharmaceutical composition to the vaginal, anal, urethral or penile glans mucosa of an individual prior to or subsequent to infection by human immunodeficiency virus.

As used herein, an ‘effective amount’ of terygyn refers to an amount that is sufficient to reduce HIV infective events in vaginal, anal, urethral and/or penile glans mucosa compared to infection with a similar number of HIV particles on untreated vaginal, anal, urethral or penile glans mucosa.

While not wishing to be bound to a specific theory, the inventor believes that terigyn acts as an adsorption inhibitor. Thus, the mechanism of action may be two fold: first, terigyn may bind directly to the gp120 glycoprotein of the HIV through the positively charged amino acids of the gp120 variable regions (V1-V3). The variable regions attract and bind to the negatively charged sulponated groups of terigyn thereby preventing the virus from attaching to host cells. The second mechanism may be through direct blocking of HIV specific receptor sites of dendritic cells (DC). Dendritic cells are involved in translocation of HIV from the exogenous through the membranes to T-cell sites. As will be known to one of skill in the art, dendritic cells are in abundance in mucosal sites, for example, the vagina, rectal, nasal and other similar regions. Terigyn hence may be in competition for the HIV carrier site in DC.

Primary peripheral blood mononuclear cells and a MT4 cell line were pretreated with varying concentrations of terygyn before infection with LAV-1, a laboratory strain of HIV-1. As a control, a set of similar cells were not pretreated with the compound. Anti-HIV activity was measured using p24 antigen testing. Terygyn achieved total HIV inhibition (IC₁₀₀) at 1 ug/ml. Cell toxicity was not observed till the concentration of terygyn was 10 ug/ml. This in vitro antiviral activity (IC₅₀ of 0.5 ug/ml) is fifty-fold more potent than that of PRO 2000, (Procept Inc) and 100 fold above that of Ushercell (Polydex Pharmaceuticals, Ontario), the two front runner microbicide candidates currently on Phase III trials. Studies on Ushercell have demonstrated that they require an IC₅₀ of 78 ug/ml to block infection with HIV (Anderson RA, et al, Journal of Andrology 2002, 23:426-438) while PRO 2000 requires 29.1 ug/ml but 1 mg/ml to completely block infection (Fletcher P et al. Retrovirology 2006, 3:46). Terygyn's ability to inhibit HIV at lower concentration makes it a more attractive compound for safe clinical use.

To further determine the inhibition of Terygyn of either CXCR4 or R5 viruses in cervical explants, various concentrations of Terygyn were cultured in TZM-bL cells with TCID50 HIV-1. TZM-bL are X4 positive HeLa cell clones that have been engineered to express CD4 and CCR5 and integrates a reporter gene for firefly luciferase and E coli b-galactosidase. Virus and drugs were left in culture for 48 hr at 37 C and luciferase assay determined thereafter. On TZM-bL cells, Terygyn inhibited the R5 viruses at IC50 of 0.13±0.05 ug/ml and X4 viruses at 0.88±1.2 ug/ml (Table 1).

In the above experiments, we found Terygyn to have anti HIV activity both in cell culture and cervical explants. However, one of our concerns was to assess the toxicity/apoptosis induction of this compound in culture to ensure the protective effect observed by a reduced p24 expression in culture was not due to a destruction of cells in culture thus limiting replicative capacity.

Terigyn was added to freshly isolated PBMC which were unstimulated at a number of different concentrations ranging from 1 to 1000 ug/ml. Cells were assessed at 5 distinct time points for the expression of phospotidyl serine as measured by annexin V binding, indicative of apoptosis and necrosis. Cells were gated on the lymphocyte population and further gated on either CD4 or CD8 expression. Each gated population was then assessed for annexin V binding.

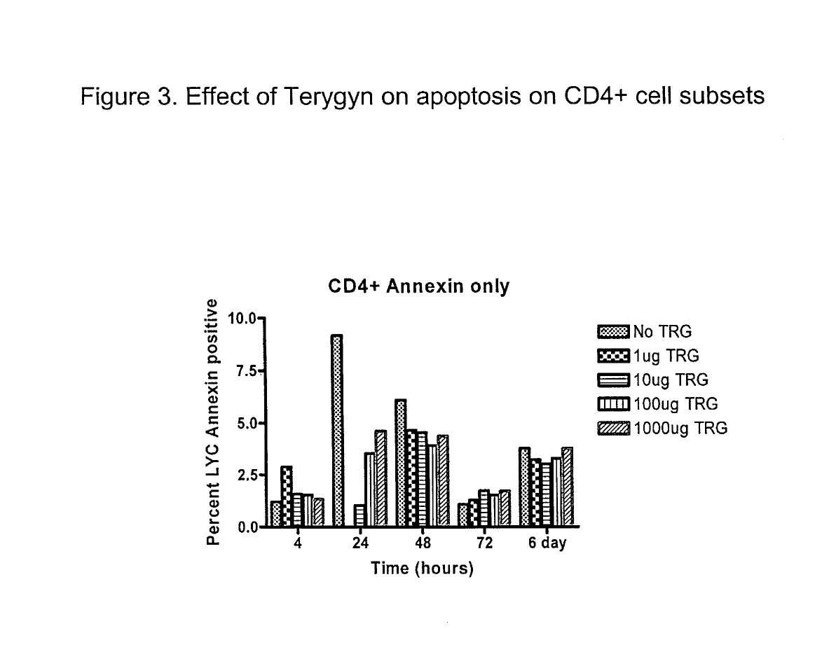

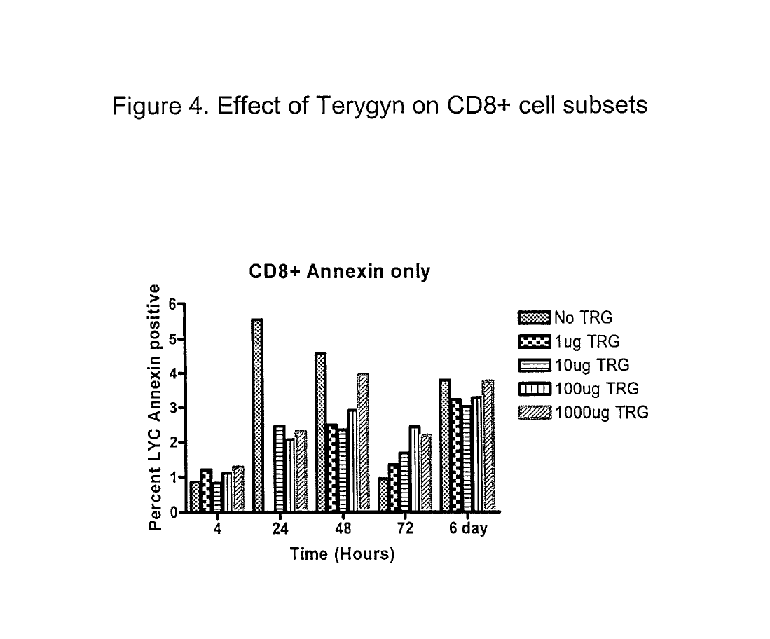

The outcome is as shown in FIGS. 3 and 4. Briefly, Terigyn reduced the amount of apoptosis among the cells particularly in the early time points (24 hrs and 48 hrs). There was also a dose response at these time points with the lower the concentration of Terygyn (1 ug-10 ug) the greater the protective effect. After this safety concentration, the higher the amount of Terygyn, the more induction of apoptosis in a stepwise manner.

It is of note that there are modified forms of terygyn which are also effective within the invention. These are the various modified cyclodextrin sulfates with various sulfonate, sulphide, and amino groups at the 2^{nd} or 6^{th} position of the cyclodextrin skeleton. These include but are not limited to mCDS3 (potassium6-deoxy-6′-0-mesitylenesulfonyl)6-pyridino-βcyclodextrin heptadecasulfate) mCDS11 potassium tris (6-benzylthio-6-dioxy) βcyclodextrin hexadecasulfate, mCDS77 (β-CD (2-0CH₂Ph)₃(SO₃K)₁₆, mCDS78 (β-CD (2-0CH₂Ph)₃(SO₃K)₉ and MCDS11 (β-CD (6-S-CH₂Ph)₃(SO₃K)₁₆.

In a preferred embodiment, the pharmaceutical composition comprises approximately 0.05% to approximately 10% terygyn or from approximately 0.49 ug/ml to approximately 9 ug/ml.

Evidence of antiviral activity by Terigyn calls for follow-up expanded laboratory and animal studies. The next step will be to carry out in vitro inhibition studies on anti-Herpes Simplex virus (HSV) and anti-human papilloma virus (anti-HPV) to be followed up by animal safety studies before it is considered for Phase I, II, III clinical trials.

While the preferred embodiments of the invention have been described above, it will be recognized and understood that various modifications may be made therein, and the appended claims are intended to cover all such modifications which may fall within the spirit and scope of the invention.

1. A method of reducing human immunodeficiency virus infective events comprising: providing a pharmaceutical composition comprising an effective amount of terygyn and a suitable excipient formulated for topical administration; and applying the pharmaceutical composition to the vaginal, anal, urethral or penile glans mucosa of an individual prior to or subsequent to infection by human immunodeficiency virus.

2. A pharmaceutical composition comprising an effective amount of terygyn and a suitable excipient for topical administration.
